(19) United States
(12) Patent Application Publication
(10) Pub. No.: US 2022/0105041 A1
(43) Pub. Date: Apr. 7, 2022

(54) METHOD FOR MANUFACTURING A SOLID ADMINISTRATION FORM AND SOLID ADMINISTRATION

(71) Applicant: MERCK PATENT GMBH, DARMSTADT (DE)

(72) Inventors: Dieter LUBDA, Bensheim (DE); Thomas KIPPING, Darmstadt (DE)

(73) Assignee: MERCK PATENT GMBH, DARMSTADT (DE)

(21) Appl. No.: 17/423,592

(22) PCT Filed: Jan. 17, 2020
(86) PCT No.: PCT/EP2020/051167

§ 371 Date: Jul. 16, 2021

(30) Foreign Application Priority Data
Jan. 18, 2019 (EP) 19152579.9

(51) Int. Cl. A61K 9/16; A61K 31/519; A61K 31/522; A61K 9/20; A61K 9/28

(52) U.S. Cl. CPC A61K 9/1694; A61K 31/519; A61K 9/1635; A61K 9/284; A61K 9/2027; A61K 9/2095; A61K 31/522

(57) ABSTRACT

For manufacturing a solid administration form comprising at least one active pharmaceutical ingredient, a flowable but setting composite material comprising the at least one active pharmaceutical ingredient is added together and set to generate the solid administration form. The flowable composite material is liquefied and delivered to a discharge unit. Small portions of liquefied composite material are intermittently discharged through an outlet into a setting unit. The flowable composite material comprises a polymer and at least one active pharmaceutical ingredient dispersed or dissolved within the polymer. The small portions are droplets and the solid administration form is generated by adding droplets that stick together before or during the setting of the liquefied composite material. An average diameter of the droplets can be less than 350 μm. There can be a void space between at least some small portions, resulting in a porous structure of the solid administration form.

### TECHNICAL FIELD

The present invention relates to a method for manufacturing a solid administration form comprising at least one active pharmaceutical ingredient, wherein a flowable but setting composite material comprising the at least one active pharmaceutical ingredient is added together and sets to generate the solid administration form.

### BACKGROUND

It is believed that future improvements in disease treatment is driven by point-of-care and home-based diagnostics linked with genetic testing and emerging technologies such as proteomics and metabolomics analysis. This has led to the concept of personalized medicine, which foresees the customization of healthcare to an individual patient. Medication can be applied to the patient by using different pharmaceutical formulations that are adapted to the desired application method, for example to oral (including buccal or sublingual), rectal, nasal, topical (including buccal, sublingual or transdermal), vaginal or parenteral (including subcutaneous, intramuscular, intravenous or intradermal) application. In general, oral application is preferred as such application is easy and convenient and does not cause any harm that may be associated with other application methods such as parenteral application. Pharmaceutical formulations usable for oral administration are, for example, capsules or tablets; powders or granules; solutions or suspensions in aqueous or non-aqueous liquids; edible foams or foam foods; or oil-in-water liquid emulsions or water-in-oil liquid emulsions. Tablets for oral administration are by far the most common dosage form and are generally prepared by either single or multiple compressions (and in certain cases with molding) processes. Tablets are usually prepared by using multiple process steps such as milling, sieving, mixing and granulation (dry and wet). Each one of these steps can introduce difficulties in the manufacture of a medicine (e.g., drug degradation and form change), leading to possible batch failures and problems in optimization of formulations.

Tablets are almost universally manufactured at large centralized plants via these processes using tablet presses essentially unchanged in concept for well over a century. This route to manufacture is clearly unsuited to personalized medicine and in addition provides stringent restrictions on the complexity achievable in the dosage form (e.g. multiple release profiles and geometries) and requires the development of dosage forms with proven long-term stability.

Usually tablets are prepared by either single or multiple compression of a prefabricated powder of an active pharmaceutical ingredient that is combined with a suitable binder agent. In most cases tablets are manufactured in large quantities at centralized manufacturing plants and afterwards distributed to the patients. However, such manufacturing does not easily allow an individual configuration of a tablet, it is not possible to adapt a tablet to needs and preferences of a single patient. Furthermore, centralized manufacture and subsequent storage and distribution to the patient requires the development of dosage forms with proven long-term stability and provides stringent restrictions on the complexity achievable in the dosage forms.

Solid administration forms are not limited to oral administration, but can also be used for other application methods, e.g. for rectal or subcutaneous administration as well as for solid forms working as release or absorber kind of devices in various application fields. However, the above described limitations of known manufacturing methods apply to most, if not all solid administration forms.

The use of additive manufacturing methods, namely 3D printing, allows for manufacture of individual solid administration forms like tablets at the point of care. Thus, a personalized tablet may be manufactured immediately before consumption by the patient. 3D printing of solid administration forms provides for many advantages, including optimized dosage of the active pharmaceutical ingredient for each patient and for each administration of a tablet, the use of individual binder agents adapted to needs or preferences of the respective patient, and individual shape and structure of the tablet resulting in a desired solubility of the tablet or different release properties of the solid administration form. The design of a customizable solid administration form like a tablet whose release is carefully controlled for individual patients and the generation on-demand using a well-known 3D printing process may support effective implementation of individualized therapy, resulting in improvements of currently applied therapy methods.

After successful testing and evaluation, there has been increased interest in the development and manufacture of 3D printing of solid administration forms after official approval of 3D printed tablets. There are many known 3D printing methods and corresponding 3D printing devices that are suitable for and used within many different fields of manufacture. These 3D printing methods include e.g. stereolithographic printing, powder bed printing, selective laser sintering, semi-solid extrusion and fused deposition modeling. Reference is made to scientific publications like, e.g. “Defined drug release from 3D-printed composite tablets consisting of drug-loaded polyvinyl alcohol and a water-soluble or water-insoluble polymer filler”, Tatsuaki Tagami et al., International Journal of Pharmaceutics 543 (2018), 361-367, Elsevier B.V. or “Adaptation of pharmaceutical excipients to FDM 3D printing for the fabrication of patient-tailored immediate release tablets”, Muzna Sadia et al., International Journal of Pharmaceutics 513 (2016), 659-668, Elsevier B.V. More general publications are related to the use of 3D printing methods for e.g. rapid prototyping of objects, including e.g. U.S. Pat. Nos. 5,204,055, 5,518,680 or EP 2 720 854 B1.

However, not all possible and known methods for 3D printing are suitable for additive manufacturing of solid administration forms with active pharmaceutical ingredients. The binder agent must meet certain requirements for 3D printing as well as for administration of the active pharmaceutical ingredients. The dosage must be well defined, reproducible for many subsequent manufacturing processes and easily controllable during manufacture of the tablet. The manufacturing process should be fast and cost effective.

Due to the increasing number of poorly water-soluble drug substances in the pipeline of research and development of pharmaceutical industry, there is a need to increase the oral bioavailability of those insoluble drug substances.

Hot-melt extrusion that is widely used in the plastics industry can be seen as a powerful technology addressing solubility of poorly soluble drugs, whereby solubility is the prerequisite of permeation of drug into a cell the bioavailability. Over the past two decades, applications of hot-melt extrusion in pharmaceutical development and drug delivery have been expanded, leading to several commercially approved products covering a variety of routes of administration.

Based on the physicochemical properties of the particular drug substance, the mechanism of bioavailability enhancement is divided into at least three categories: formation of amorphous solid dispersions, formation of crystalline solid dispersions, and formation of co-crystals.

Formulation of amorphous solid dispersions is a viable approach for improving the dissolution performance of poorly water-soluble drug substances. It is especially suitable for non-ionizable drug substances that cannot form pharmaceutical salts. The amorphous drug substance is stabilized within the matrix in order to prevent any re-crystallization.

Amorphous drug exists in a higher energy state than crystalline drugs, and this can result in higher kinetic solubility and a faster dissolution rate. This allows drug molecules present in amorphous solid dispersions to be more readily absorbed from the gastrointestinal tract.

In order to increase the rate of dissolution it is well known to prepare formulations of poorly soluble compounds in form of solid dispersions.

Various processes can be used to create solid dispersions. In general, these systems can be produced by processes either utilizing solvents or which require the melting of one or more of the added substances. These solid dispersions can be created by a number of methods, including, but not limited to, spray-drying, melt extrusion and thermokinetic compounding. A recently applied technology to support solubility of poor soluble drugs is the deposition of the drug in amorphous phase onto a carrier, e.g. porous silica.

Both melt extrusion and spray drying processes are widely used to prepare amorphous solid dispersions to enhance bioavailability of biopharmaceutics classification system classes II and IV drugs.

To achieve an amorphous dispersion through spray drying, for example, the solvent or co-solvent system utilized must be suitable to dissolve both the polymeric carrier vehicle and the compound of interest. In summary, these methods require the use of a solvent system, often organic in nature, to dissolve an inert carrier and active drug substance (Serajuddin A. T. M.; Solid dispersion of poorly water-soluble drugs: early promises, subsequent problems, and recent breakthroughs. J Pharm Sci. (1999), 88 (10), 1058-1066). Once a solution is formed, the solvent is subsequently removed by a mass transfer mechanism dependent on the manufacturing technique chosen. Although solvent-based techniques such as spray drying are relatively common, they suffer from several disadvantages. Selection of a solvent system that is compatible with the active substance and carrier polymer may prove to be difficult or require very large amounts of organic solvent. This presents a safety hazard at the manufacturing facility as organic solvents must be collected and disposed of properly to limit the environmental impact.

It is currently considered and widely accepted that fused deposition modelling seems to be the most promising approach for 3D printing of solid administration forms like tablets or capsules or implants. The use of fused deposition modelling for additive manufacturing tablets as well as the required preparation of a suitable filament that is fed to the 3D printer which generates the tablet is described e.g. in “Coupling 3D printing with hot-melt extrusion to produce controlled-release tablets”, Jiaxian Zhang et al., International Journal of Pharmaceutics 519 (2017), 186-197, Elsevier B.V.

However, manufacturing the filament from a mixture of a suitable binder agent and the one or several active pharmaceutical ingredients is laborious, but required for fused deposition modelling. Manufacturing the active pharmaceutical ingredients containing filament is much more complicated as of standard polymer filaments, as the active pharmaceutical ingredients must be introduced into the binder agent, usually a suitable polymer or composite material, in a stabilized crystalline or in its amorphous form to enhance the solubility and as a result also the bioavailability of the active pharmaceutical ingredient. The characteristics of the binder agent must allow for producing and storing the filament within a wound up and spools form. This usually requires the addition of plasticizer or stabilizer into the binder agent, which may interfere with the health safety of the filament from which the tablet is produced. Thus, use of the fused deposition modelling method for manufacture of solid administration forms imposes severe restrictions on the choice and preparation of suitable materials for the binder agent and the active pharmaceutical ingredients.

Accordingly, there is a need for a method for manufacturing a solid administration form that can be performed easily and cost-effectively, but also allows for personalized manufacture of single solid administration forms.

### SUMMARY OF THE INVENTION

The present invention relates to a method for manufacturing a solid administration form comprising at least one active pharmaceutical ingredient, wherein a flowable but setting composite material comprising the at least one active pharmaceutical ingredient is added together and sets to generate the solid administration form, whereby the flowable composite material is liquefied and delivered to a discharge unit, and whereby small portions of the liquefied composite material are intermittently discharged through an outlet of the discharge unit into a setting unit where the setting of small portions occurs, thereby gradually generating the solid administration form. This manufacturing method of claims 1 to 11 allows for additive manufacturing with known 3D printing devices, but does not require the tedious prefabrication of a filament that is fed to the 3D printing device. Rather, the composite material that comprises a binder agent as well as the active pharmaceutical ingredient can be granules prepared by different methods as hot melt extrusion, wet granulation, dry compaction, twin screw granulation. It is also possible to make use of a mixture of different material or compositions in particulate form of active pharmaceutical ingredients and binder agents that form a mixture with suitable flowability that is prepared immediately before delivery to the discharge unit. Granules and such particle mixtures are much easier to prepare compared to a filament. Co-milling processing can be used in order to achieve a homogenous distribution of pharmaceutical ingredients and binder agents prior to processing.

There is no need to meet diameters and restrictions related to the dimensions and flexibility of a filament. Furthermore, granules, particles and other kinds of mixtures or single components before mixing are easier to store and less susceptible to chemical and mechanical stress during storage and transport, if required. As there is no need for prefabrication of a filament, there is no need for another melting of the composite material and subsequent manufacture of the filament. In case of a preparation of the composite material just before delivery of the composite material to the discharge unit, it is possible to make use of crystalline or amorphous forms of active pharmaceutical ingredients to create solid dispersions or solid solutions with improved solubility of otherwise poorly soluble active pharmaceutical ingredients. Contrary to fused deposition modelling, it is easily possible to add crystalline or non-soluble active pharmaceutical ingredients or other non-soluble additives into the composite material.

Furthermore, there is no need for laborious preparation and in particular for melting and subsequent setting of the composite material. Thus, it is also possible to make use of active pharmaceutical ingredients with a melting point above the melting point of the corresponding binder agent.

Examples of application fields for advantageous use of the invention include, but are not limited to, disease treatment by point-of-care, personalized medicine by customization of healthcare to an individual patient, cost effective preparation of small batch sizes of final administration forms or for drugs with limitation in product storage. Small and flexible batch sizes are needed to deliver a product for clinical phases supply. It also simplifies the use of several different formulation forms from pre-clinic to final approval by establishing generic formulation processes, which might speed-up registration processes due to the faster approval of final drugs. The invention also allows for formulation of orphan drugs or commercial offering of final administration forms containing high toxic compounds as well as at point-of-care e.g. for cancer treatment in clinics. Products with higher drug load, i.e. higher content of active pharmaceutical ingredients are possible in comparison by using other methods to prepare solid administration forms.

The core of invention offers pharmaceutical industry tools to address trends in personalization of medicine very much related to geriatrics and pediatrics. Option to offer in special for elderly people product cocktails containing different drugs, i.e. enhanced customer convenience, focusing on generic use and very easy adoption to tablet size needed in pediatrics. Tablet sizes in diameter of 1 mm to 6 mm, a challenge to prepare by common technologies, could be prepared accordingly. Additional manufacturing advantages of invention include continuous manufacturing processing could be connected much easier as possible so far, flexibility from a modular setup, and easy scale-up. Final appearance of administration form depending size, design and outer and internal form could be prepared very flexible as well.

A suitable binder agent may comprise pharmaceutically acceptable excipients known to those skilled in the art, which may be used to produce the composites and compositions disclosed herein. Examples of excipients for use with the present invention include, but are not limited to, e.g., a pharmaceutically acceptable polymer, or a non-polymeric excipient. Other non-limiting examples of excipients include, lactose, glucose, starch, calcium carbonate, kaoline, crystalline cellulose, silicic acid, water, simple syrup, glucose solution, starch solution, gelatin solution, carboxymethyl cellulose, shellac, methyl cellulose, polyvinyl pyrrolidone, dried starch, sodium alginate, powdered agar, calcium carmelose, a mixture of starch and lactose, sucrose, butter, hydrogenated oil, a mixture of a quaternary ammonium base and sodium lauryl sulfate, glycerine and starch, lactose, bentonite, colloidal silicic acid, talc, stearates and polyethylene glycol, sorbitan esters, polyoxyethylene sorbitan fatty acid esters, polyoxyethylene alkyl ethers, poloxamers (polyethylene-polypropylene glycol block copolymers), sucrose esters, sodium lauryl sulfate, oleic acid, lauric acid, polyoxyethylated glycolyzed glycerides, dipalmitoyl phosphadityl choline, glycolic acid and salts, deoxycholic acid and salts, cyclodextrins, polyethylene glycols, polyglycolyzed glycerides, polyvinyl alcohols, polyvinyl acetates, polyvinyl alcohol/polyethylene glycol graft copolymer, polyacrylates, polymethacrylates, polyvinylpyrrolidones, phosphatidyl choline derivatives, cellulose derivatives, biocompatible polymers selected from poly-(lactides), poly(glycolides), poly(lactide-co-glycolides), poly(lactic acid)s, poly(glycolic acid)s, poly(lactic acid-coglycolic acid)s and blends, combinations, and copolymers thereof.

Selection of the polymer carrier system is considered important for the successful development of formulation and manufacturing processes. The physicochemical and mechanical properties of polymers and drug substances must be carefully evaluated.

As both a thermal and mechanical process, hot-melt extrusion applies a significant amount of heat and shear stresses on the materials being subjected to the hot-melt extrusion process. As a result, the drug substances and the polymeric carriers may undergo chemical reactions.

Therefore, the chemical properties and the stability of the formulation components must be monitored in order to eliminate any degradation concerns. The chemical reactions are divided into the main chain reactions and the side chain reactions. The main chain reactions comprise the chain scission and cross-linking; while the side chain reactions comprise the side chain elimination and the side chain cyclization.

Suitable thermal binder agents that may or may not require a plasticizer include, for example, Eudragit® RS PO, Eudragit® SIOO, Kollidon® SR (Polyvinyl acetate-Polyvinylpyrrolidone mixture), Kollidon® VA 64 (vinylpyrrolidone-vinyl acetate copolymers), Kollicoat IR (polyvinyl alcohol/polyethylene glycol graft copolymer), Soluplus® (polyvinyl caprolactam-polyvinyl acetate-polyethylene glycol graft copolymer), Ethocel® (ethylcellulose), HPC (hydroxypropylcellulose), cellulose acetate butyrate, poly(vinylpyrrolidone) (PVP), poly(ethylene glycol) (PEG), poly(ethylene oxide) (PEO), poly(vinyl alcohol) (PV A), hydroxypropyl methylcellulose (HPMC), ethylcellulose (EC), hydroxyethylcellulose (HEC), sodium carboxymethyl-cellulose (CMC), dimethylaminoethyl methacrylate-methacrylic acid ester copolymer, ethylacrylate-5 methylmethacrylate copolymer (GA-MMA), C-5 or 60 SH-50 (Shin-Etsu Chemical Corp.), cellulose acetate phthalate (CAP), cellulose acetate trimelletate (CAT), poly(vinyl acetate) phthalate (PV AP), hydroxypropylmethylcellulose phthalate (HPMCP), poly(methacrylate ethylacrylate) (1:1) copolymer (MA-EA), poly(methacrylate methylmethacrylate) (1:1) copolymer (MA-MMA), poly(methacrylate methylmethacrylate) (1:2) copolymer, Eudragit® L-30-D (MA-EA, 1:1), 10 Eudragit® L-100-55™ (MA-EA, 1:1), Eudragit® E (EPO) (copolymer based on dimethylaminoethyl methacrylate, butyl methacrylate, and methyl methacrylate), hydroxypropylmethylcellulose acetate succinate (HPMCAS), Coateric® (PV AP), Aquateric® (CAP), and AQUACOAT® (HPMCAS), polycaprolactone, starches, pectins; polysaccharides such as tragacanth, gum arabic, guar gum, and xanthan gum.

A binary dispersion of an active pharmaceutical ingredient and a binder agent can exist as a single-phase system, or as a multi-phase system, depending on their miscibility. In general, a single-phase amorphous solid dispersion system is desired for the following reasons. First of all, a single-phase system tends to have better stability compared to a multiphase system. Due to phase separation, multi-phase systems comprise a drug-rich domain and a polymer-rich domain. In most cases, the drug-rich domain has a relatively low glass transition temperature and the drug molecules are less protected. Therefore, the drug-rich domain is more susceptible to re-crystallization, raising a physical stability concern. Regarding the drug substance that has good physical stability in the amorphous state, phase separation may negatively impact the dissolution performance of the formulation. A water-soluble polymer matrix facilitates the dissolution process of a poorly-soluble drug substance.

Yet another embodiment of the present invention includes a method of pre-plasticizing one or more pharmaceutical polymers by blending the polymers with one or more plasticizer selected from the group consisting of oligomers, copolymers, oils, organic molecules, polyols having aliphatic hydroxyls, ester-type plasticizers, glycol ethers, poly(propylene glycols), multi-block polymers, single block polymers, poly(ethylene oxides), phosphate esters; phthalate esters, amides, mineral oils, fatty acids and esters thereof with polyethylene-glycol, glycerin or sugars, fatty alcohols and ethers thereof with polyethylene glycol, glycerin or sugars, and vegetable oils by mixing prior to agglomeration, by processing the one or more polymers with the one or more plasticizers into a composite

Examples of active pharmaceutical ingredients either approved or new and under development include, but are not limited to, antibiotics, analgesics, vaccines, anticonvulsants; antidiabetic agents, antifungal agents, antineoplastic agents, antiparkinsonian agents, antirheumatic agents, appetite suppressants, biological response modifiers, cardiovascular agents, central nervous system stimulants, contraceptive agents, dietary supplements, vitamins, minerals, lipids, saccharides, metals, amino acids and precursors, nucleic acids and precursors, contrast agents, diagnostic agents, dopamine receptor agonists, erectile dysfunction agents, fertility agents, gastrointestinal agents, hormones, immunomodulators, anti-hypercalcemia agents, mast cell stabilizers, muscle relaxants, nutritional agents, ophthalmic agents, osteoporosis agents, psychotherapeutic agents, para-sympathomimetic agents, para-sympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents. In certain embodiments, the active pharmaceutical ingredient is a poorly water-soluble drug or a drug with a high melting point. The active pharmaceutical ingredient may be found in the form of one or more pharmaceutically acceptable salts, esters, derivatives, analogs, prodrugs, and solvates thereof.

According to an aspect of the invention the flowable composite material comprises a polymer and at least one amorphous active pharmaceutical ingredient that is mechanically mixed, dispersed or dissolved with or within the polymer. For many pharmaceutical applications a poor solubility or bioavailability of active pharmaceutical ingredients is addressed with hot melt extrusion of the composite material, which allows for incorporation of the active pharmaceutical ingredients in its amorphous forms into the polymer. However, contrary to fused deposition modelling there is no need to create a filament that is immediately afterwards coiled onto a spool, which causes mechanical stress and quite often reduces the desired solubility of the active pharmaceutical ingredients within the composite material, e.g. during storage of the coiled filaments on the spool. Furthermore, there is also no need to stabilize the amorphous forms within the filament in order to preserve the amorphous forms during subsequent unwounding and feeding of the filament to the discharge unit of a fused deposition modelling printer, which again causes mechanical stress and instability to the filament by creating more fragile areas within the composite material. Also, for crystalline forms of active pharmaceutical ingredients, the reduced thermal stress and the only once performed transfer into its amorphous form during melting until discharge of the small portions of the composite material significantly enhances the solubility and bioavailability of poorly soluble active pharmaceutical ingredients. The risk for polymorphic transitions during a potential second heating step is therefore avoided.

In yet another embodiment of the invention the flowable but setting composite material includes non-soluble porous or non-porous carrier particles for altering or enhancing the properties of the solid administration form. By adding carrier particles it is possible to improve the solubility of the active pharmaceutical ingredient applied. Furthermore, added carrier particle can change release properties or stabilize the active pharmaceutical ingredient against thermal degradation during the manufacturing process.

According to an advantageous embodiment of the invention, the flowable composite material is fabricated during delivery to the discharge unit, i.e. very shortly or immediately before the intermittently discharge of liquefied small portions of the composite material with the discharge unit. Thus, there will be no degradation of the active pharmaceutical ingredients and/or of the composite material due to long term storage of the composite material or due to transport of the prefabricated composite material to the discharge unit. It is also possible to make use of granules that are heated and liquefied immediately before delivery to the discharge unit. Alternatively, a mixture of particles can be used to generate the composite material by heating and melting the mixture of particles and subsequently delivering the molten mixture of the particle generated composite material to the discharge unit.

For many applications, there is no need for addition of stabilizing materials into the composite material, as there is no need for long-term storage of the prefabricated composite material until final use of the composite material for additive manufacturing of a solid administration form. However, for some applications it might be advantageous to add stabilizer and/or plasticizer to the composite material in order to adapt the properties and in particular mechanical properties of the composite material and the resulting solid administration form to individual requirements of the respective applications.

According to another favorable aspect of the invention the small portions of the liquefied composite material are droplets and that the solid administration form is generated by adding droplets that bond or stick together before or during the setting of the liquefied composite material. Intermittently discharging droplets of fluids is a well-known method e.g. for administration of the fluid onto a surface during ink printing processes. Intermittently discharging a liquefied composite material is similar to those methods and it is possible for a person skilled in the art to make use of suitable devices in order to create a solid administration form by arranging discharged and subsequently solidified droplets into the desired shape of the solid administration form. Contrary to fused deposition modelling there is no continuous filament that imposes restrictions on the additive generation of objects like continuous deposition of composite material along uninterrupted deposition lines.

Furthermore, it is possible to modify the properties and e.g. the porosity of the solid administration form and thus it's disintegration as well as the solubility and bioavailability of the active pharmaceutical ingredient therein by presetting and controlling the bonding or sticking together of the respective small portions or droplets that are intermittently discharged to generate the solid administration form. The less closely linked the single small portions or droplets are after final setting of the composite material, the more porous is the resulting solid administration form. It is also possible to vary the porosity of the solid administration form within the volume of the solid administration form.

According to an advantageous embodiment of the invention an average diameter of the droplets is less than 350 μm, preferably less than 200 μm. The smaller the size of a single droplet, the more complex shapes and structures of the solid administration form are possible and can be additively generated with great precision. In order to be able to manufacture solid administration forms comprising a reasonable large volume of composite material in a reasonably short period of time, the size of a single droplet should be larger than 20 μm and preferably larger than 50 μm. In another embodiment of the invention the preparation of structures of the solid administration forms prepared from different average diameters of the droplets can lead to structures with unique properties not possible to prepare using other technologies. As it seems possible to discharge several 100 droplets per second through a single nozzle of the discharge unit, a fairly rapid generation of tablets and similar solid administration forms is possible. Furthermore, a small diameter of a single droplet enables the generation of tablets with an individual, but well-defined content of the active pharmaceutical ingredient or ingredients. In another embodiment of the invention an average diameter of the droplets is larger than 350 μm if the function of the administration form and the containing active pharmaceutical ingredients is not influenced by a resulting faster preparation.

In yet another embodiment of the invention there is a void space between at least some small portions that are placed adjacent to each other, resulting in a porous structure of the solid administration form. As the solid administration form is composed of a large number of small portions of the composite material, whereby each small portion is separately discharged from the discharge unit, there is no limitation with respect to the respective position of adjacent small portions or droplets. Thus, the distance between adjacent small portions or droplets can be preset in order to either generate a very dense, homogeneous and uniform solid administration form or to generate a filigree and porous structure with many void spaces between adjacent portions of the composite material within the solid administration form.

According to another embodiment of the invention the small portions of the composite material are discharged into an arrangement of the small portions such that the solid administration form comprises at least two regions with different characteristics of the active pharmaceutical ingredient. As explained before, by making use of the method according to this invention it is not necessary to generate the solid administration form by applying a continuous filament to the generated base body of the solid administration form. Contrary thereto, each small portion can be placed at will and at a predetermined distance to the last or next discharged small portion. Thus, it is easily possible to manufacture a solid administration form that is inhomogeneous or comprises sections with different structure or composition within a single solid administration form.

According to another aspect of the invention, before or after discharging a predetermined first amount of a composite material a predetermined second amount of a second material is discharged, whereby the material of the second material differs from the composite material. Thus, it is also possible to make use of two or more different composite materials within a single solid administration form. For example, a porous structure of a first composite material with a poorly or rapidly soluble active pharmaceutical ingredient may be encased with a surrounding layer of a binder agent without any active pharmaceutical ingredient in order to e.g. prepare solid administration forms with preset shielding properties, decorative or taste masking or with predefined enteric properties. The first and second composite material can be delivered to and discharged from the discharge unit one after another, making use of the same means for delivering and discharging the composite material. In addition, depending on the manufacturing device there may be further discharging units, which can be provided with differently composed mixtures to be used in combination with first composite material. This means, that the manufacturing device can include the numbers of discharging units are more than two and can be different in nozzles diameter.

That is, the manufacturing device may have more than one or two discharging units. In addition, the discharging units may have different cross-sections, so that the size of dispensed composite units may be different in a time unit and thus the internal structure of the product produced may be different depending on the units used and the compositions discharged per unit.

However, in order to enhance manufacturing speed and to reduce undesired contamination of the respective composite material that is used to generate some parts of a solid administration form it is considered advantageous to provide for separate delivering and discharging means for each different composite material that is used for the additive manufacturing of a single solid administration form. For example, the discharge unit may comprise separate delivery channels that feed into a dedicated nozzle of the discharge unit, whereby each delivery channel and corresponding nozzle can be activated and used separately.

Varying the porosity or composition of the solid administration form within the volume of the solid administration form, e.g. creating a gradient of active pharmaceutical ingredients within the volume of the solid administration form allows for enhanced control of solubility and bioavailability of the active pharmaceutical ingredients over long terms of administration. Thus, it is possible to generate solid administration forms as implants for subcutaneous administration and long-term deposition that will dispense a preset and constant amount of active pharmaceutical ingredients for weeks, months and even for years.

It is considered advantageous to provide for a rigidly mounted discharge unit that is arranged over a manufacturing plate or table that can be moved with respect to the discharge unit. The manufacturing plate can be an XY-table that can be arbitrarily translated within a plane. It is also possible to vary the distance between the manufacturing plate and the outlet of the discharge unit resulting in the use of a XYZ-table, e.g. to adapt to the height and top surface of the additively manufactured solid administration form that step by step grows during the manufacturing process.

Of course, it is also possible to provide for a discharge unit with several means for discharging the composite material at the same time, thus manufacturing several solid administration forms at the same time. The discharge unit may comprise several nozzles that are connected to the same or separate means for delivering the liquefied composite material to the nozzles.

The invention also relates to a solid administration form comprising at least one active pharmaceutical ingredient.

According to an aspect of the invention, the solid administration form is manufactured by liquefying a flowable composite material and delivering the liquefied composite material to a discharge unit, whereby small portions of the liquefied composite material are intermittently discharged through an outlet of the discharge unit into a setting unit where the setting of small portions occurs, thereby gradually generating the solid administration form. By discharging a predetermined number of small portions of the composite material that comprises the active pharmaceutical ingredient, it is possible to precisely define the content of the active pharmaceutical ingredient within the solid administration form for each sample. Thus, the solid administration form is not defined by macroscopic characteristics like e.g. weight or dimension, but even more precisely defined by the number and spatial arrangement of the small portions that have been subsequently discharged to additively manufacture the solid administration form.

According to an advantageous embodiment of the invention the solid administration form comprises small portions of two different composite materials. The small portions of the first and second composite material can be arranged in separate but adjacent regions within the solid administration form. It is also possible to arrange for a homogeneous distribution of first and second small portions of the respective first and second composite material. Furthermore, the composite material with the active pharmaceutical ingredient can be coated with a material without any active pharmaceutical ingredient that only provides for pleasant taste during oral administration of the solid administration form.

In yet another embodiment of the invention the density of small portions of the composite material within the solid administration form varies between different regions within the solid administration form. It is possible to encompass a porous inner region with a dense casing or coating, whereby a mean distance between the respective center of adjacent small portions in the porous inner region is larger than a mean distance between the respective center of adjacent small portions in the dense casing or coating. It is also possible to create a gradient of density, i.e. a gradient of mean distance between the center of adjacent small portions that varies from the inner middle to the outer surface of the solid administration form.

Furthermore and according to an advantageous aspect of the invention, it is possible to create solid administration forms with hollow structures, e.g. mesh-like structures with void spaces inside the solid administration form. Thus, it is possible to adapt the solubility and bioavailability of the active pharmaceutical ingredient within the solid administration form according to individual needs and personal preferences.

According to yet another embodiment of the invention the small portions comprised within the solid administration form are separate droplets of composite material, whereby the droplets are arranged adjacent to each other and connected via connecting surfaces during setting of the liquefied composite material.

### DETAILED DESCRIPTION OF THE INVENTION

FIG. 1 illustrates a schematic view of a manufacturing device 1 for additive manufacturing of a solid administration form 2. The manufacturing device 1 comprises a discharge unit 3 with a nozzle 4 that is directed towards a manufacturing platform 5 mounted on top of a XY-table 6. With the help of the XY-table 6 the manufacturing platform 5 can perform translation movements in two directions perpendicular to a discharging direction 7 of the nozzle 4 of the discharge unit 3. It is also possible to provide for a height adjustment of the XY-table 6, i.e. to make use of a XYZ-table. This allows for controlling and adjusting the distance between the nozzle 4 and the surface of the manufacturing platform 5 during the additive manufacture of the solid administration form 2.

The manufacturing device 1 also comprises a storage container 8 that can be filled with basic raw materials like polymer granules prepared by different technologies or even particle and fluid like materials and active pharmaceutical ingredients using a feed hopper 9 or feeding lines 10 (gravimetric dosing devices can be added in order to further increase the precision). The storage container 8 is connected via a screw conveyor 11 with the discharge unit 3. According to different embodiments of the invention the screw conveyor 11 can be a single-screw extruder with smooth or grooved barrel, a twin-screw extruder with co-rotating or counterrotating screws as well as with intermeshing or non-intermeshing screws, or a multi-screw extruder with static or rotating central shaft with the general potential to use adjustable screw geometry. The basic raw materials are fed to the discharge unit 3 through the screw conveyor 11. Within the screw conveyor 11 or discharge unit 3 the basic raw materials are mixed together, homogenized and liquefied into a composite material. It is also possible to add heat optional with a temperature control in order to adjust the targeted temperature profile. Different heating sections can be used in order to achieve a homogenous melt and transport to the discharge unit 3 or to the screw conveyor 11 in order to support the liquefication of the composite material. The composite material is intermittently discharged through the nozzle 4 onto the manufacturing platform 5. Each small portion 12 that is discharged through the nozzle 4 connects with other small portions 12 and solidifies to gradually generate the solid administration form 2.

The shape and dimension of the solid administration form 2 are determined by the number of small portions 12 that are discharged through the nozzle 4 and by the movement of the XY-table during the discharge of the small portions 12. Optional several nozzles 4 with different diameter (generating separate droplets of composite material with different average diameter) can be used. The content of the active pharmaceutical ingredient deposited within the solid administration form 2 is determined by the content of the active pharmaceutical ingredient within the composite material and by the number of small portions 12 that are discharged during manufacturing of the solid administration form 2. Thus, by presetting the total number of small portions 12 that are added, composed and solidified for the additive generation of the solid administration form 2 the total content of the active pharmaceutical ingredient can be precisely and individually controlled for each solid administration form 2 that is generated by using the manufacturing device 1.

The manufacturing platform 5 can be enclosed inside a housing that provides for controlled manufacturing conditions with respect to e.g. temperature, illumination or humidity. The manufacturing platform 5 and the housing as well as controlling devices for the manufacturing conditions are part of a setting unit 13 that allows for controlling the setting of the previously liquefied small portions 12 of the composite material in order to create the desired shape and structure of the solid administration form 2.

FIGS. 2, 3 and 4 illustrate a schematic perspective view of three different embodiments of the solid administration form 2 that is each composed of a large number of small portions 12 of composite material. Each small portion 12 is a single droplet of the composite material that comprises at least one suitable polymer material and at least one active pharmaceutical ingredient.

The solid administration form 2 shown in FIG. 2 is composed of a very large number of small portions 12 that are arranged very close next to each other, thereby creating a very dense and approximately homogeneous solid body after successive solidification of the small portions 12. The mean diameter of the small portions 12 is preferably more than 50 μm but less than 150 μm, and the frequency of the intermittently discharged small portions 12 is between approx. 50 and 150 droplets per second. Even though the duration of solidification of a single small portion 12 is quite short, each following small portion 12 fuses together with the small portions 12 already discharged before, thus generating a very homogeneous body of the solid administration form 2. The duration of the solidification of the small portions 12 can be controlled e.g. by transferring heat or cold to the manufacturing platform 5 or a manufacturing space above the top of the manufacturing platform 5. It is also possible to make use of a composite material that comprises a polymer that is susceptible to e.g. UV light illumination or electricity which may enhance or delay the solidification process.

The solid administration form 2 shown in FIG. 3 is composed of a smaller number of small portions 12 compared to the solid administration form 2 of FIG. 2. The mean diameter of the small portions 12 is larger than in FIG. 2, whereby the small portions 12 have a mean diameter of e.g. approx. 350 μm. The small portions 12 are arranged at a small distance to each other, thereby generating a porous solid administration form 2. The density of the composed solid administration form 2 is significantly less than the density of the solid administration form 2 shown in FIG. 2. The mean distance between adjacent small portions 12 is similar to the mean diameter of the small portions 12. The porosity and density of the solid administration form 2 is to a large extend adjustable at will by presetting the mean diameter of the small portions 12 and the mean distance of adjacent small portions 12.

FIG. 4 schematically illustrates a solid administration form 2 comprising void spaces 14 within the solid administration form 2. The void spaces 14 are created by introducing a mean distance between some adjacent small portions 12 that is larger than the mean diameter of the small portions 12. Furthermore, the frequency of discharging subsequent small portions 12 can be adapted in order to allow for at least some setting of the previously discharged small portion 12 resulting in improved mechanical stability of the already generated part of the solid administration form 2 before adding a following small portion 12 at a predetermined position of the already generated part of the solid administration form 2. Contrary to conventional compression molding of tablets, the creation of void spaces 14 is easily achieved by controlling the movement of the XY-table during additive manufacturing of the solid administration form 2. When compared to known additive manufacturing methods like e.g. fused deposition modelling, the method according to the present invention allows for more variations of the arrangement of the small portions 12 that are intermittently discharged during the manufacturing process, resulting in more complex shapes and structures of solid administration forms 2.

FIGS. 5 and 6 illustrate a schematic perspective view and a sectional view of another embodiment of a solid administration form 2. Within a middle region 15 of the solid administration form 2 a first number of small portions 12 of a first composite material 16 have been arranged and connected with each other. A second number of small portions 17 of a second material 18 encompasses the middle region 15, thereby creating an encasement 19 of the middle region 15. Only the first composite material 16 in the middle region 15 comprises the active pharmaceutical ingredient, whereas the second material 18 delays the absorption of the first composite material 16 with the active pharmaceutical ingredient. Thus it is possible to generate a solid administration form 2 having a repository effect for the active pharmaceutical ingredient that can be predetermined by the composition and thickness of the encasement 19 of the second material.

FIGS. 7 and 8 schematically illustrate yet another embodiment of a solid administration form 2. Beginning in the middle of the solid administration form 2, the solid administration form 2 is composed of two different first and second composite materials 16, 20, whereby alternating layers of either the first composite material 16 or the second composite material 20 create respective encasements for the enclosed inner parts of the solid administration form 2. The first composite material 16 and the second composite material 20 comprise different active pharmaceutical ingredients. This allows for an alternating absorption of two different active pharmaceutical ingredients during the dissolution of the solid administration form 2. Additional variations resulting in more complex shapes and structures of solid administration forms 2 with the option to generate different properties (e.g. fast, slow, targeted or other kind of release of the active pharmaceutical ingredient).

FIGS. 9 and 10 schematically illustrate an embodiment of the solid administration form 2 similar to the embodiments shown in FIGS. 5 and 6, but with a very thin encasement 19 of the second material 18 with a thickness of only one or few small portions 17 that encloses the large middle region 15 with the first composite material 16 comprising the active pharmaceutical ingredient. The thin encasement 19 of the second material 18 can be used e.g. for masking the taste of the first composite material 16 or for adding a gliding surface, which in both cases increases the acceptance of the patients for oral administration of the solid administration form 2.

FIGS. 11 and 12 schematically illustrate another embodiment of the solid administration form 2, whereby several layers of the first composite material 16 are bonded together with interjacent arranged layers of the second material 18.

FIG. 13 illustrates a section view of yet another embodiment of the solid administration 2 form with a density of adjacent small portions 12 increasing from the middle region 15 to an outer surface 21 of the solid administration form 2. FIG. 14 illustrates a section view of yet another embodiment of the solid administration form 2 with a density of adjacent small portions 12 decreasing from the middle region 15 to the outer surface 21 of the solid administration form 2.

The above described manufacturing method also allows for manufacturing of solid administration forms 2 with complex shapes and structures. By way of example, FIGS. 15 and 16 schematically illustrate a top view of such complex embodiments of the solid administration form 2 with a ring-shaped outer structure 22 and with an cross-shaped structure 23 inside the ring-shaped outer structure 22. There are large void spaces 24 arranged inside of the ring-shaped outer structure 22 that enhances the quick dissolution of the solid administration form 2. It is possible to create the solid administration form 2 out of the same first composite material 16, as shown in FIG. 15, or to make use of two or three different first, second and third composite materials 16, 20 and 25 with either different content of the same active pharmaceutical ingredient or with different active pharmaceutical ingredients, as shown in FIG. 16. It is also possible to include parts or structural elements made of a second material 18 without active pharmaceutical ingredients.

FIGS. 17 and 18 schematically illustrate yet another embodiment of the solid administration form 2 composed of five strip-shaped structures each comprising a different composite material 16, 20, 25, 26 and 27.

FIGS. 19, 20 and 21 schematically illustrate exemplary embodiments of complex shapes for the solid administration form 2. FIG. 19 shows a ball-shaped hollow solid administration form 2 with a mesh-like casing 28, FIG. 20 illustrates a tablet-shaped solid administration form 2, and FIG. 21 illustrates a torus-shaped solid administration form 2.

### LIST OF FIGURES

FIG. 1: Schematic view of a manufacturing device for additive manufacturing of a solid administration form.

FIG. 2: Schematic perspective view of a solid administration form composed of a large number of small portions of composite material.

FIG. 3: Schematic perspective view of another embodiment of a solid administration form composed of larger small portions that the embodiment shown in FIG. 2.

FIG. 4: Schematic perspective view of another embodiment of a solid administration form comprising void spaces within the solid administration form.

FIG. 5: Schematic perspective view of another embodiment of a solid administration form.

FIG. 6: Section view of the solid administration form shown in FIG. 5 along the line VI-VI in FIG. 5.

FIG. 7: Schematic perspective view of another embodiment of a solid administration form.

FIG. 8: Section view of the solid administration form shown in FIG. 7 along the line VIII-VIII in FIG. 7.

FIG. 9: Schematic perspective view of another embodiment of a solid administration form.

FIG. 10: Section view of the solid administration form shown in FIG. 9 along the line X-X in FIG. 9.

FIG. 11: Schematic perspective view of another embodiment of a solid administration form.

FIG. 12: Top view of the solid administration form shown in FIG. 11

FIG. 13: Section view of yet another embodiment of a solid administration form with a density of adjacent small portions increasing from the middle to the outer surface of the solid administration form

FIG. 14: Section view of yet another embodiment of a solid administration form with a density of adjacent small portions decreasing from the middle to the outer surface of the solid administration form.

FIG. 15: top view of yet another embodiment of a solid administration form with a ring-shaped outer structure and with a cross-shaped structure inside the ring-shaped outer structure.

FIG. 16: top view of yet another embodiment of a solid administration form similar to the embodiment shown in FIG. 15 but comprising three different composite materials.

FIG. 17: side view of yet another embodiment of a solid administration form composed of five strip-shaped structures each comprising a different composite material.

FIG. 18: top view of the solid administration form shown in FIG. 17

FIG. 19: Schematic perspective view of another embodiment of a ball-shaped hollow solid administration form with a mesh-like casing.

FIG. 20: Schematic perspective view of another embodiment of a tablet-shaped or capsule-shaped solid administration form

FIG. 21: Schematic perspective view of another embodiment of a torus-shaped solid administration form.

FIG. 22: Example 7: 3D printed tablet comprising pure PVA as suitable thermal binder with 100% filling rate.

FIG. 23: Example 8: 3D printed tablet comprising a binary dispersion of PVA as suitable thermal binder and 10% Caffeine as active pharmaceutical ingredient with 100% filling rate.

FIG. 24: Example 9; 3D printed tablet comprising a binary dispersion of PVA and 10% Caffeine with 50% filling rate.

FIG. 25: Example 10; 3D printed tablets comprising a binary dispersion of PVA and 10% Dipyridamole with 100% filling rate.

FIG. 26: Example 11; 3D printed tablets comprising a binary dispersion PVA and 10% Dipyridamole with 50% filling rate.

FIG. 27: Example 12; 3D printed tablets comprising a binary dispersion of PVA and 10% Dipyridamole with 30% filling rate.

FIG. 28: Example 13: 3D printed tablets with outer shell (100% filling rate) of pure PVA and an inner core comprising a binary dispersion of PVA as suitable thermal binder and dipyridamole (yellow/orange color) as active pharmaceutical ingredient. Printing was stopped after 2 mm height for better visibility of principle.

FIG. 29: Example 13; 3D printed tablets with outer shell (50% filling rate) of pure PVA and an inner core comprising a binary dispersion of PVA as suitable thermal binder and dipyridamole (yellow/orange color) as active pharmaceutical ingredient

FIG. 30: Release of Dipyridamole: Results achieved by dissolution measurement of 3D printed dipyridamole containing tablets (Ex. 10, 11 and 12) in phosphate buffer pH 6.8

FIG. 31: Release of Caffeine: Results achieved by dissolution measurement of 3D printed caffeine containing tablets (Ex. 8 and Ex. 9) in 0.1 n HCl.

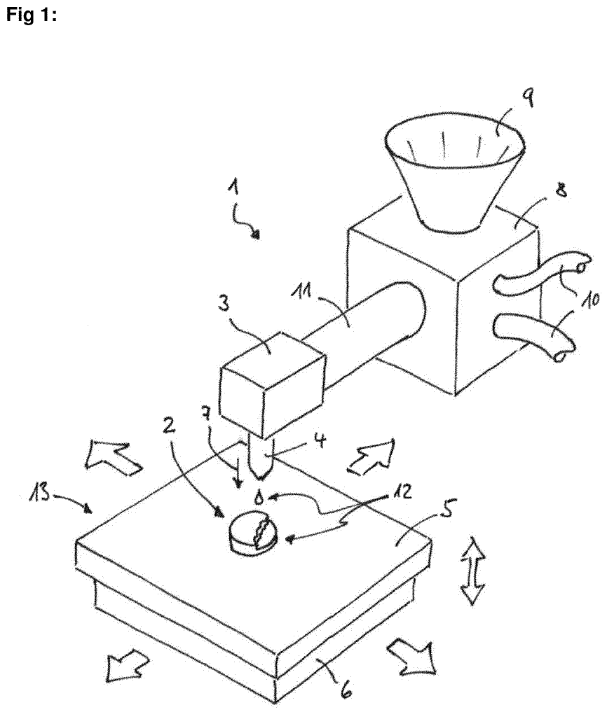

### EXAMPLES

The present description enables the person skilled in the art to apply the invention comprehensively. Even without further comments, it is assumed that a person skilled in the art will be able to utilize the above description in the broadest scope.

Practitioners will be able, with routine laboratory work, using the teachings herein, to prepare active ingredients comprising formulations as defined above in the new process.

### Example 1

### Preparation of a Suitable Thermal Binder in Form of Granules, to be Used in the 3D Printing Process, by Hot Melt Extrusion (HME)

Pre-treatment of the material:

For the preparation of a suitable thermal binder in form of granules for the 3D Printing process by HME 2.0 kg polyvinyl-alcohol=PVA (Parteck MXP, Cat No 141360 from Merck KGaA Germany) with optimized particle size distribution for HME is dried at 85° C. in a vacuum oven.

Extrusion is started by adjusting the dosing rate of the dosing unit and the screw speed of the extruder in small increments until the target parameters of 0.35 kg/h and 350 rpm reached. This takes about 5 minutes from starting the process until the first exit of extrudate from the nozzle. The extrudate emerges as very homogeneous, transparent strand from the nozzle (2 mm in diameter), having a yellow-orange color.

Extruder conditions used:

Pressure at the nozzle 14-15 bar.

Melting temperature 192° C. and a torque of 41-42%,

Heating zones HZ 1=80° C./HZ 2−HZ 7=200° C.

Nozzle temperature=200° C.

The extrudate strand is discarded for about 10 minutes until it emerges homogeneously from the die. Thereafter, the strand is started to be conveyed to the pelletizer by means of a conveyor belt, which gives the extrudate a short cooling phase at room temperature and then it is cut into 1.5 mm pellets in length. The material is finally dried under vacuum conditions at 85° C. before it is used in 3D printing device to a LOD<0.1%.

### Example 2

### Preparation of a Binary Dispersion Comprising Dipyridamole as Active Pharmaceutical Ingredient (API) and PVA as Thermal Binder in Form of Granules for Use in the 3D Printing Process, by Hot Melt Extrusion (HME)

Preparation of the mixture:

The binary mixture of PVA polymer (dried at 85° C. in a vacuum oven) and 10% API is prepared by mixing of 1.8 kg of PVA 4-88 (Parteck MXP, Cat No 141360 from Merck KGaA Germany) and 0.2 kg Dipyridamole Ph. Eur (LGM Pharma) as model API with yellow colour in a 10 L drum using a Röhnradmischer for 15 minutes.

Extrusion is started by adjusting the dosing rate of the dosing unit and the screw speed of the extruder in small increments until the target parameters of 0.35 kg/h and 350 rpm reached. This takes about 5 minutes from starting the process until the first exit of extrudate from the nozzle. The extrudate emerges as very homogeneous, transparent strand from the nozzle (2 mm in diameter), having a yellow-orange colour.

Extruder conditions:

Pressure at the nozzle 14-15 bar.

Melting temperature 192° C. and a torque of 41-42%,

Heating zones HZ 1=80° C./HZ 2−HZ 7=200° C.

Nozzle temperature=200° C.

The extrudate strand is discarded for about 10 minutes until it emerges homogeneously from the die. Thereafter, the strand is started to be conveyed to the pelletizer by means of a conveyor belt, which gives the extrudate a short cooling phase at room temperature and then it is cut into 1.5 mm pellets in length. The material is finally dried under vacuum conditions at 85° C. before use in 3D printing device to a LOD<0.1%.

### Example 3

### Preparation of a Suitable Thermal Binder for Use in the 3D Printing Process, by “Dry Compaction”

For the preparation of a suitable thermal binder in form of dry compacted granules for the 3D Printing process 2.6 kg polyvinyl-alcohol (PVA; Parteck MXP, Cat No 141360 from Merck KGaA Germany) are compacted by a physical dry compaction process.

For the dry compaction process a Powtec-Kompaktor RCC 100x20 (Powtec Maschinen und Engineering GmbH, Remscheid, Deutschland) is used, equipped with a sieve of 2.24 mm mesh size. The product introduction of PVA powder is carried out with 30 rpm. For compaction, lumbers provided with lines and a lumber speed of 3 rpm a hydraulic pressure of 125 bars with a lumber slit of 2.1 mm as well as a sieving mill speed of 50 rpm is used.

Dry compacted PVA 4-88 granules (>710 μm) are prepared with a yield of 2.28 kg under conditions as described before. The material is finally dried under vacuum conditions at 85° C. before use in 3D printing device to a LOD<0.1%.

### Example 4

### Preparation of a Binary Dispersion of an API and PVA as Suitable Thermal Binder in Form of Granules, to be Used in the 3D Printing Process, by “Dry Compaction”

Preparation of the mixture:

The binary mixture of PVA polymer and 10% API is prepared by mixing 1.8 kg of PVA 4-88 (Parteck MXP, Art No 141360 from Merck KGaA Germany) with 0.2 kg Caffeine (from Shandong Xinhua Pharmaceuticals China) as model API in a 12 L drum using a Röhnradmischer Elte 650, (Engelsmann AG, Ludwigshafen, Deutschland) for 5 minutes (36 rpm). After the first mixing time the mixture of PVA polymer and caffeine are homogenized by using a 710 μm sieve followed by another 5 minutes of mixing.

For dry compaction 1.9 kg of the resulting mixture is dry compacted using a Powtec-Kompaktor RCC 100x20 (Powtec Maschinen und Engineering GmbH, Remscheid, Deutschland), equipped with a sieve of 2.24 mm mesh size. Product introduction of PVA powder is carried out with 30 rpm. For compaction, lumbers provided with lines and a lumber speed of 3 rpm a hydraulic pressure of 125 bars with a lumber slit of 1.5 mm as well as a sieving mill speed of 50 rpm is used.

Resulting dry compacted mixture with a yield of 1.66 kg of PVA 4-88/caffeine granules (>710 μm) prepared using conditions as described before. The material is finally dried under vacuum conditions at 85° C. before use in 3D printing device to a LOD<0.1%.

### Example 5

### Preparation of a Suitable Thermal Binder for Use in the 3D Printing Process, by Twin Screw Wet Granulation (TSG)

Granulation:

1.6 kg of PVA 4-88 (Parteck MXP, Cat. No 141360, Merck KGaA Germany) are weighed into a stainless-steel bowl and sieved through a 1 mm sieve into a 5 L stainless-steel barrel and mixed for 10 min in a drum hoop mixer.

For the granulation a Pharma 11 hot melt extruder modified with a TSG conversion kit (ThermoFisher Scientific) is used. The powder mixture is added with a gravimetric feeder (Brabender Congrav OP1T), and DI water is added with a peristaltic pump (Cole-Parmer Masterflex L/S). Each screw consists of 4 Long Helix Feed Screws 3/2 UD, 4 Feed Screws 1 L/D, 7 mixing elements 60° offset, 26 Feed Screws 1 L/D, 1 Distributive Feed Screw (front to end).

Before granulation, the barrel temperature is set to 30° C. Then the barrel is flooded with water at slow screw speed (10 rpm) and a water addition of ˜200 mL/h. To prepare the granules the water addition is reduced to 30.1 mL/h, which corresponds to the L/S ratio of 0.086. The screw speed is increased to 50 rpm and powder addition is started with an amount of 0.1 kg/h. Then the screw speed and the powder feed-rate are increased stepwise (50-, then 100 rpm steps) until the desired screw speed of 500 rpm is reached and the powder feed-rate is increased up to a feed rate of 0.35 kg/h (0.05 kg/h steps).

The first material processed in this manner is discarded. When the torque has reached a constant level (after approx. 5 min) the resulting granules are collected in a stainless-steel bowl. To get the desired amount of 1 kg granules, the granulation is run for almost 3 hours. Resulting granules are tray dried in a vacuum oven for 24 h at 50° C./0.1 bar to a LOD<0.1%.

Before use in the 3D printing process material the product is additionally sieved through a 5 mm sieve in order to avoid a blocking of the dosing of granules into the 3D printer by contained coarse particles.

### Example 6

### Preparation of a Binary Dispersion of an API and PVA by Twin Screw Wet Granulation as Suitable Thermal Binder for Use in the 3D Printing Process

a) Preparing the mixture:

1.6 kg of PVA 4-88 (Parteck MXP, Cat. No 141360, Merck KGaA Germany) and 0.4 kg of Dipyridamole Ph. Eur (LGM Pharma) are weighed into a stainless-steel bowl. Then both components are sieved through a 1 mm sieve into a 5 L stainless-steel barrel and mixed for 10 min in a drum hoop mixer.

b) Granulation:

For the granulation process a Pharma 11 hot melt extruder is used modified with a TSG conversion kit (ThermoFisher Scientific). The powder mixture is added with a gravimetric feeder (Brabender Congrav OP1T) DI water is added with a peristaltic pump (Cole-Parmer Masterflex L/S). Each screw consisted of 4 Long Helix Feed Screws 3/2 L/D, 4 Feed Screws 1 L/D, 7 mixing elements 60° offset, 26 Feed Screws 1 L/D, 1 Distributive Feed Screw (front to end).

Before granulation, the barrel temperature is set to 30° C. Then the barrel is flooded with water at slow screw speed (10 rpm) and a water addition of ˜200 mL/h. To prepare the granules the water addition is reduced to 30.1 mL/h, which corresponds to the L/S ratio of 0.086. Then the screw speed is increased to 50 rpm and the powder addition is started with 0.1 kg/h. the screw speed and the powder feed-rate are increased stepwise until the desired screw speed of 500 rpm (50-, then 100 rpm steps) and a powder feed-rate of 0.35 kg/h (0.05 kg/h steps) are reached.

The first material processed in this manner is discarded. When the torque has reached a constant leave (after approx. 5 min) the resulting granules are collected in a stainless-steel bowl. To get the desired amount of 1 kg granules, the granulation is run for almost 3 hours. The resulting granules are tray dried in a vacuum oven for 24 h at 50° C./0.1 bar to a LOD<0.1%.

Before use in the 3D printing process the material is additionally sieved through a 5 mm sieve in order to avoid a blocking of the dosing of granules into the 3D printer by contained coarse particles.

c) 3 D printing process using a suitable thermal binder as composite material with and without addition of API:

The process of printing is performed whereby the flowable composite material is liquefied and delivered to a discharge unit, whereby small portions of the liquefied composite material are intermittently discharged through an outlet of the discharge unit into a setting unit where the setting of small portions occurs, thereby gradually generating the solid administration form. This manufacturing method of additive manufacturing does not require the tedious prefabrication of a filament that is fed to the 3D printing device.

The suitable thermal binder as pure polymer or mixtures of polymer and API additives prepared in examples 1-6 are used for the printing of solid administration forms in an additive manufacturing process (3D Printing) with a “Freeformer” from ARBURG GmbH+Co KG, Lossburg, Germany.

### Example 7

### 3D Printing of Tablets of Pure PVA as Suitable Thermal Binder with 100% Filling Rate

The suitable thermal binder in granulated form, prepared in Example 1, with a material density of 1.27 g/cm³ was pre-dried before feeding into the printing device. The residual moisture (goal<0.5%) is measured with an Aquatrac gauge at a temperature of 120° C. with 0.32%.

When the preconditioned, granulated material which is prepared in Examples 1, is used, neither bridging nor feeding problems are observed throughout the experimental series

### Evaluation of Printing Parameter and Printing of Solid Administration Form

a) Determination of processing parameters & discharge properties:

Granulated material, which is prepared in Examples 1, forms well separable droplets, and homogeneously drops out from the nozzle. At a nozzle temperature of 220° C. the material shows translucent droplets. The required drop height of 200 μm+10-20% was achieved with 70% discharge.

b) Conditions used for the printing process:

Temperature discharge unit: 200° C.

Temperature zone 2: 190° C.

Temperature zone 1: 180° C.

Temperature printing room: 80° C.

Dynamic pressure: 40 bar

Metering stroke: 6 mm

Decompression speed: 2 mm/s

Decompression space: 5 mm

Discharge: 70%

In order to find the suitable aspect ratio, test printing with different slicer volume (ratio of width and layer thickness) is adjusted. Best properties can be achieved with an aspect ratio of 1.36 using a material as prepared in Example 1.

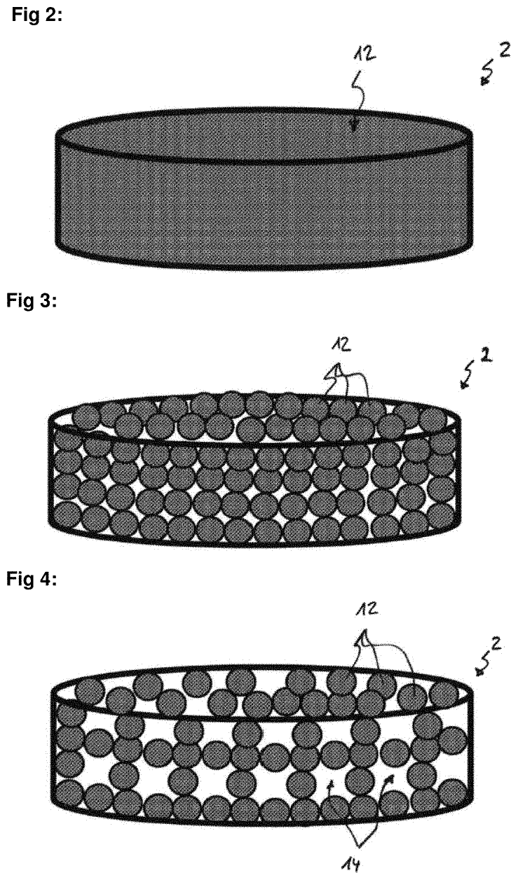

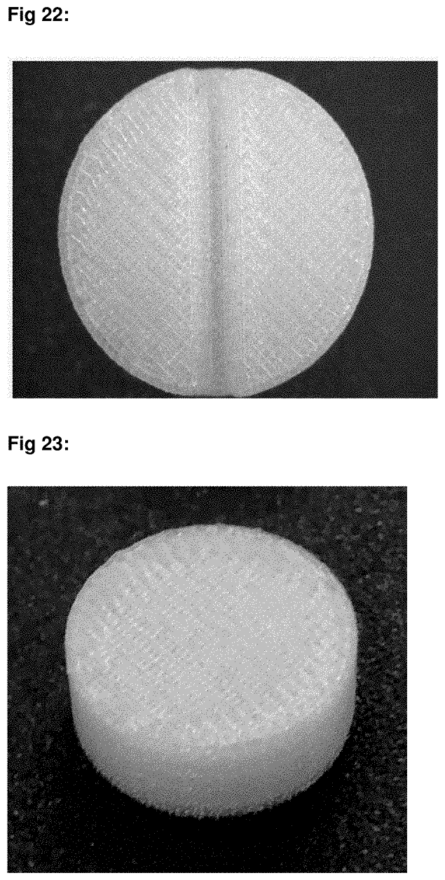

If conditions are used as described before and if the binder of Example 1 is used an optimized 3D printing process can be performed to generate the solid administration form as projected and depicted in FIG. 2. Resulting solid administration form with 100% filling rate of polyvinyl alcohol was analyzed by optical method (FIG. 22).

### Example 8

### 3D Printing of Tablets of Binary Dispersion PVA as Suitable Thermal Binder and 10% Caffeine as Active Pharmaceutical Ingredient with 100% Filling Rate

The suitable thermal binary binder (PVA+10% caffeine) in granulated form, prepared in Example 4, are pre-dried before feeding into the printing device. The residual moisture (goal<0.5%) is measured with an Aquatrac gauge at a temperature of 120° C. with 0.07%.

Using the preconditioned granulated material prepared in Examples 4 neither bridging nor feeding problems are observed throughout the experimental series
Evaluation of printing parameter and printing of solid administration form:

### Determination of Processing Parameters & Discharge Properties

Granulated material prepared in Examples 4 formed well separable droplets, homogeneously dropping out from the nozzle. At a nozzle temperature of 200° C. the material shows translucent droplets. The required drop height of 200 μm+10-20% was achieved with 65% discharge.

Conditions used for the printing process:

Temperature discharge unit: 190° C.

Temperature zone 2: 180° C.

Temperature zone 1: 170° C.

Temperature printing room: 80° C.

Dynamic pressure: 80 bar

Metering stroke: 5 mm

Decompression speed: 2 mm/s

Decompression space: 5 mm

Discharge: 65%

In order to find the suitable aspect ratio, test printings with different slicer volume (ratio of width and layer thickness) are adjusted. Best properties can be achieved with an aspect ratio of 1.34 using material prepared in Example 4.

By using conditions describe before, optimize 3D printing process is performed with suitable binder of Example 4 (polyvinyl alcohol+10% caffeine) to generate the solid administration form as projected and depicted in FIG. 2. Resulting solid administration form with 100% filling rate of the binder mixture of polyvinyl alcohol+10% caffeine as API is analyzed by optical method (FIG. 23).

### Example 9

### 3D Printing of Tablets of Binary Dispersion PVA as Suitable Thermal Binder and 10% Caffeine as Active Pharmaceutical Ingredient with 50% Filling Rate

The suitable thermal binary binder (PVA+10% caffeine) in granulated form, prepared in Example 4, is pre-dried before feeding into the printing device. The residual moisture (goal<0.5%) is measured with an Aquatrac gauge at a temperature of 120° C. with 0.07%.

Using the preconditioned granulated material prepared in Example 4 neither bridging nor feeding problems are observed throughout the experimental series

Evaluation of printing parameter and printing of solid administration form:
Determination of processing parameters and discharge properties

Granulated material prepared in Examples 4 form well separable droplets, homogeneously dropping out from the nozzle. At a nozzle temperature of 200° C. the material shows translucent droplets. The required drop height of 200 μm+10-20% is achieved with 65% discharge.
Conditions used for the printing process:

Temperature discharge unit: 190° C.

Temperature zone 2: 180° C.

Temperature zone 1: 170° C.

Temperature printing room: 80° C.

Dynamic pressure: 80 bar

Metering stroke: 5 mm

Decompression speed: 2 mm/s

Decompression space: 5 mm

Discharge: 65%

In order to find the suitable aspect ratio, test printings with different slicer volume (ratio of width and layer thickness) are adjusted. Best properties can be achieved with an aspect ratio of 1.34 using material prepared in Example 4.

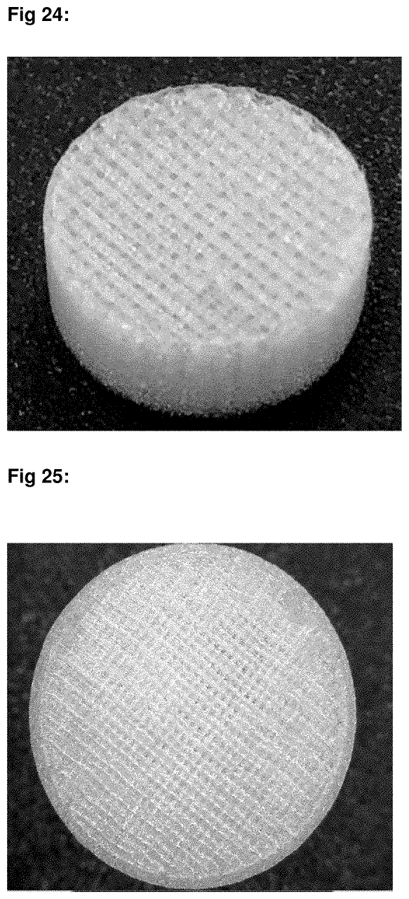

By using conditions as described before, an optimized 3D printing process is performed with a suitable binder of Example 4 (polyvinyl alcohol+10% caffeine) to generate the solid administration form as projected and depicted in FIG. 3. Resulting solid administration form with 50% filling rate of binder mixture polyvinyl alcohol+10% caffeine as API is analyzed by an optical method (FIG. 24).

### Example 10

### 3D Printing of Tablets of Binary Dispersion PVA as Suitable Thermal Binder and 10% Dipyridamole with 100% Filling Rate

The suitable thermal binary binder (PVA+10% Dipyridamole) in granulated form, prepared in Example 2, is pre-dried before feeding into the printing device. The residual moisture (goal<0.5%) is measured with an Aquatrac gauge at a temperature of 120° C. with 0.28%.

Using the preconditioned granulated material prepared in Example 2 neither bridging nor feeding problems are observed throughout the experimental series

Evaluation of printing parameter and printing of solid administration form:
Determination of processing parameters and discharge properties

Granulated material prepared in Example 2 forms well separable droplets, homogeneously dropping out from the nozzle. At a nozzle temperature of 200° C. the material shows translucent droplets. The required drop height of 200 μm+10-20% is achieved with 65% discharge.
Conditions used for the printing process:

Temperature discharge unit: 190° C.

Temperature zone 2: 170° C.

Temperature zone 1: 160° C.

Temperature printing room: 80° C.

Dynamic pressure: 80 bar

Metering stroke: 6 mm

Decompression speed: 2 mm/s

Decompression space: 5 mm

Discharge: 65%

In order to find the suitable aspect ratio, test printings with different slicer volume (ratio of width and layer thickness) are adjusted. Best properties can be achieved with an aspect ratio of 1.31 using material prepared in Example 2.

By using conditions described before, optimized 3D printing process is performed with suitable binder of Example 2 (polyvinyl alcohol+10% Dipyridamole) to generate the solid administration form as projected and depicted in FIG. 2. Resulting solid administration form with 100% filling rate of binder mixture polyvinyl alcohol+10% Dipyridamole as API is analyzed by an optical method (FIG. 25).

### Example 11

### 3D Printing of Tablets of Binary Dispersion PVA as Suitable Thermal Binder and 10% Dipyridamole with 50% Filling Rate

The suitable thermal binary binder (PVA+10% Dipyridamole) in granulated form, prepared in Example 2, is pre-dried before feeding into the printing device. The residual moisture (goal<0.5%) is measured with an Aquatrac gauge at a temperature of 120° C. with 0.28%.

Using the preconditioned granulated material prepared in Example 2 neither bridging nor feeding problems are observed throughout the experimental series

Evaluation of printing parameter and printing of solid administration form:
Determination of processing parameters & discharge properties

Granulated material prepared in Example 2 forms well separable droplets, homogeneously dropping out from the nozzle. At a nozzle temperature of 200° C. the material shows translucent droplets. The required drop height of 200 μm+10-20% is achieved with 65% discharge.
Conditions used for the printing process:

Temperature discharge unit: 190° C.

Temperature zone 2: 170° C.

Temperature zone 1: 160° C.

Temperature printing room: 80° C.

Dynamic pressure: 80 bar

Metering stroke: 6 mm

Decompression speed: 2 mm/s

Decompression space: 5 mm

Discharge: 65%

In order to find the suitable aspect ratio, a test printing with different slicer volume (ratio of width and layer thickness) is adjusted. Best properties can be achieved with an aspect ratio of 1.31 using material prepared in Example 2.

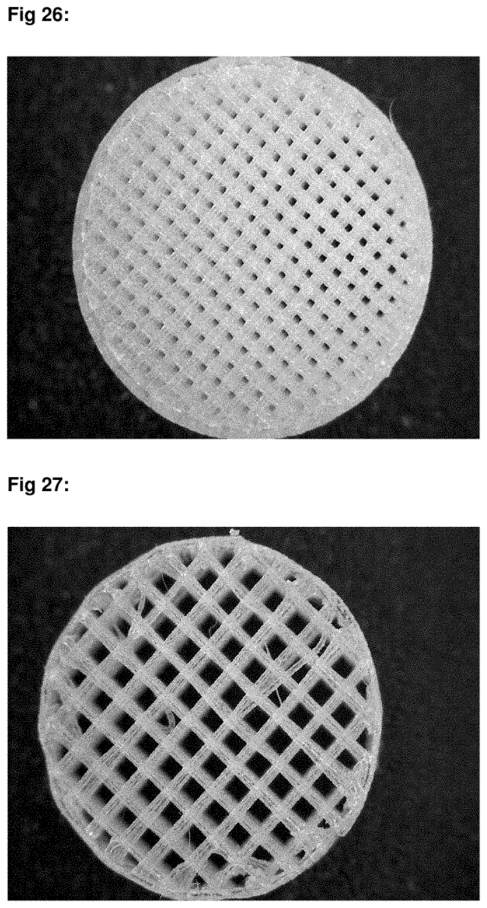

By using conditions as described before, optimized 3D printing process is performed with suitable binder of Example 2 (polyvinyl alcohol+10% Dipyridamole) to generate the solid administration form as projected and depicted in FIG. 3. Resulting solid administration form with 50% filling rate of binder mixture polyvinyl alcohol+10% Dipyridamole as API is analyzed by optical method (FIG. 26).

### Example 12

### 3D Printing of Tablets of Binary Dispersion PVA as Suitable Thermal Binder and 10% Dipyridamole with 30% Filling Rate

The suitable thermal binary binder (PVA+10% Dipyridamole) in granulated form, prepared in Example 2, is pre-dried before feeding into the printing device. The residual moisture (goal<0.5%) is measured with an Aquatrac gauge at a temperature of 120° C. with 0.28%.

Using the preconditioned granulated material prepared in Example 2 neither bridging nor feeding problems are observed throughout the experimental series

Evaluation of printing parameter and printing of solid administration form:
Determination of processing parameters & discharge properties

Granulated material prepared in Example 2 forms well separable droplets, homogeneously dropping out from the nozzle. At a nozzle temperature of 200° C. the material shows translucent droplets. The required drop height of 200 μm+10-20% is achieved with 65% discharge.
Conditions used for the printing process:

Temperature discharge unit: 190° C.

Temperature zone 2: 170° C.

Temperature zone 1: 160° C.

Temperature printing room: 80° C.

Dynamic pressure: 80 bar

Metering stroke: 6 mm

Decompression speed: 2 mm/s

Decompression space: 5 mm

Discharge: 65%

In order to find the suitable aspect ratio, a test printing with different slicer volume (ratio of width and layer thickness) is adjusted. Best properties can be achieved with an aspect ratio of 1.31 using material prepared in Example 2.

By using conditions described before, an optimized 3D printing process is performed with suitable binder of Example 2 (polyvinyl alcohol+10% Dipyridamole) to generate the solid administration form as projected and depicted in FIG. 4. Resulting solid administration form with 30% filling rate of binder mixture polyvinyl alcohol+10% Dipyridamole as API is analyzed by an optical method (FIG. 27).

### Example 13

### 3D Printing of Tablets with Outer Shell (100% Filling Rate) of Pure PVA and an Inner Core of a Binary Dispersion PVA as Suitable Thermal Binder and Dipyridamole (Yellow/Orange Color) as Active Pharmaceutical Ingredient

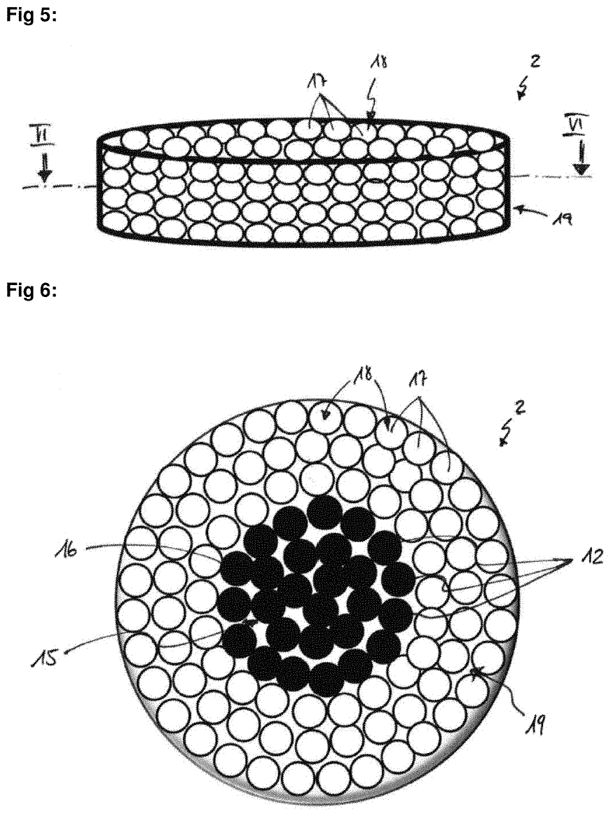

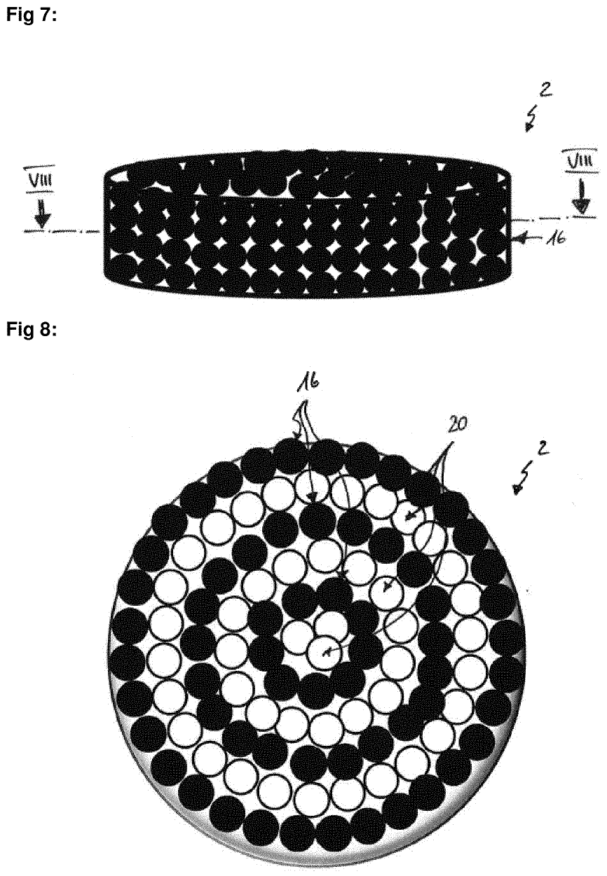

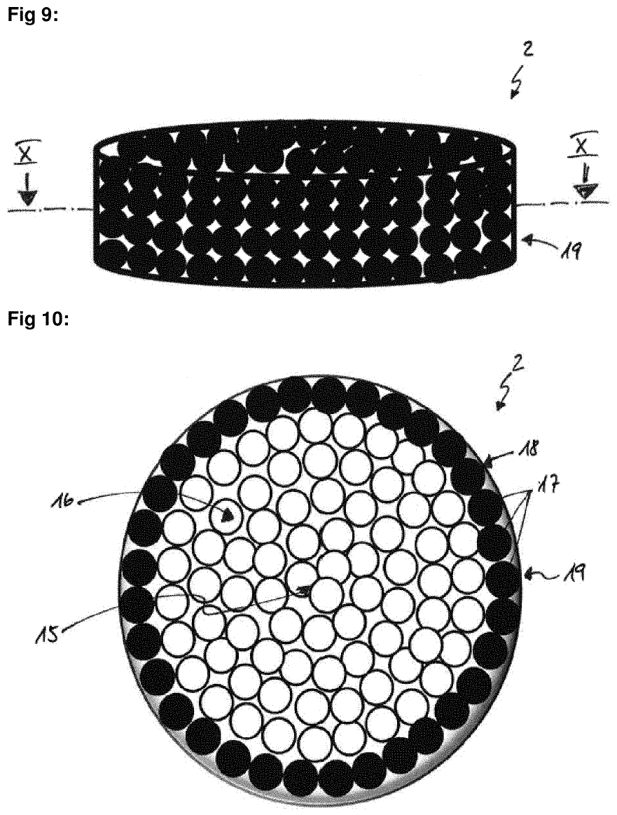

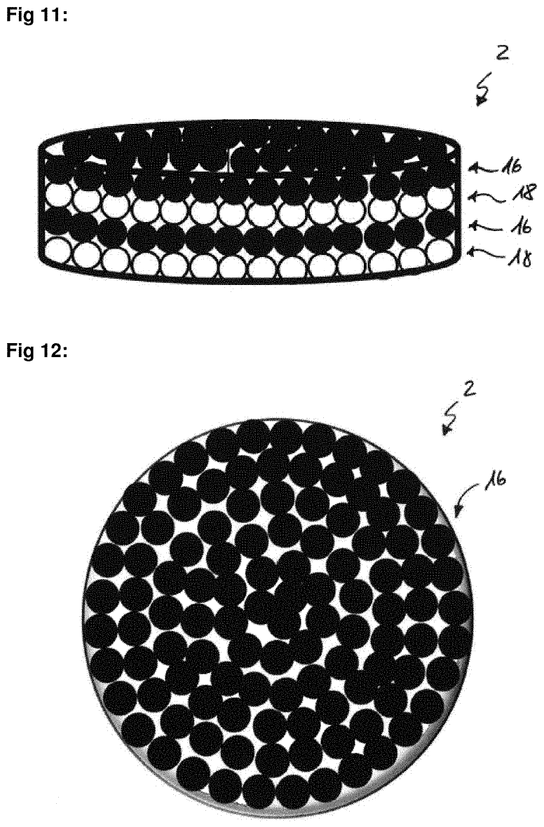

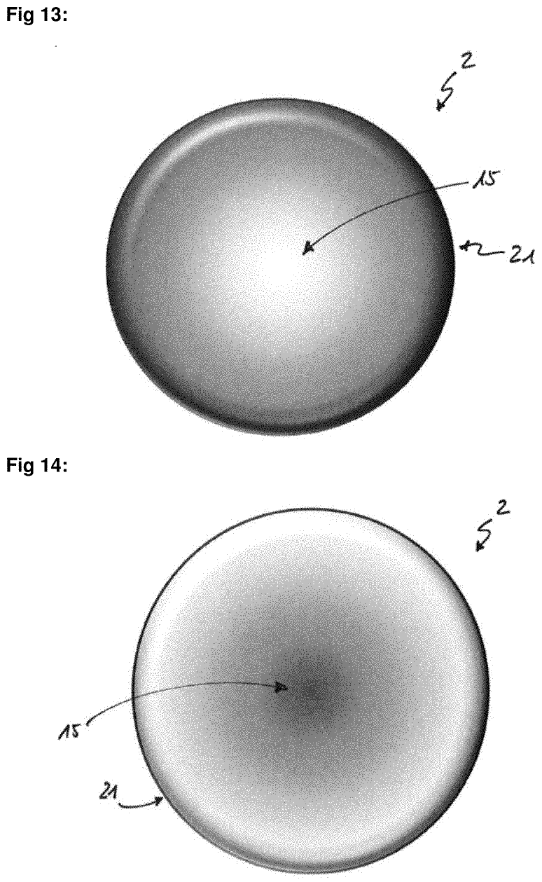

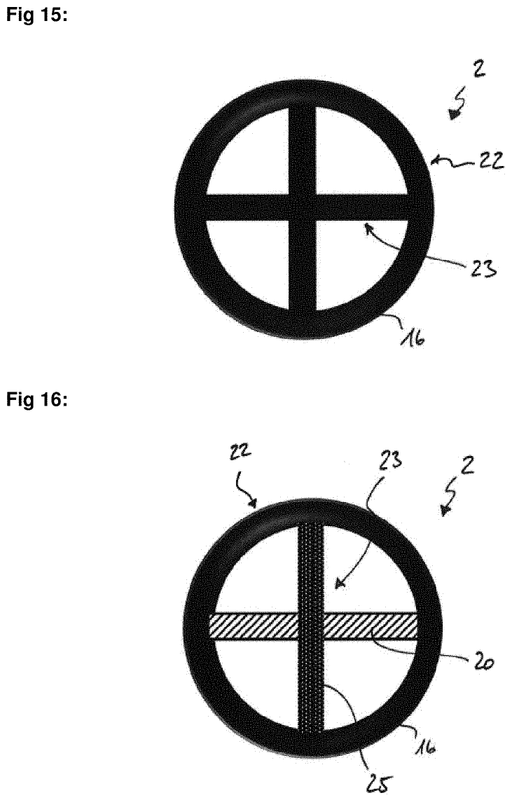

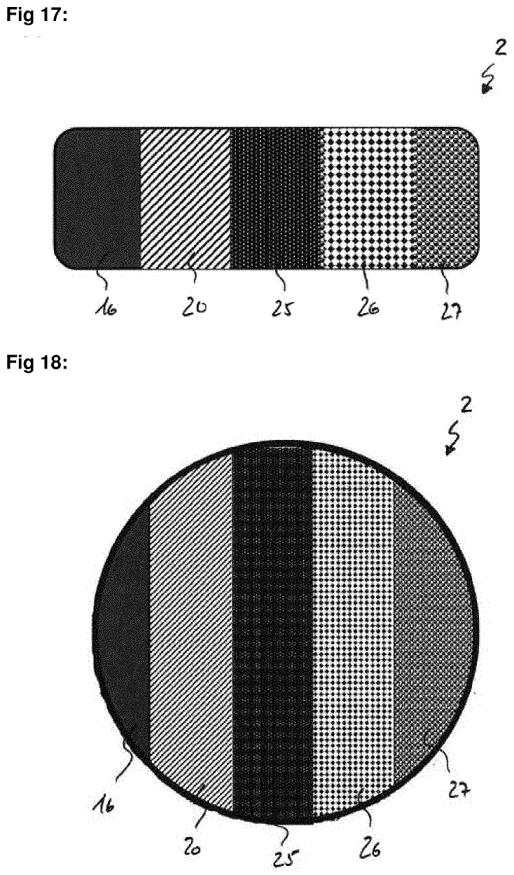

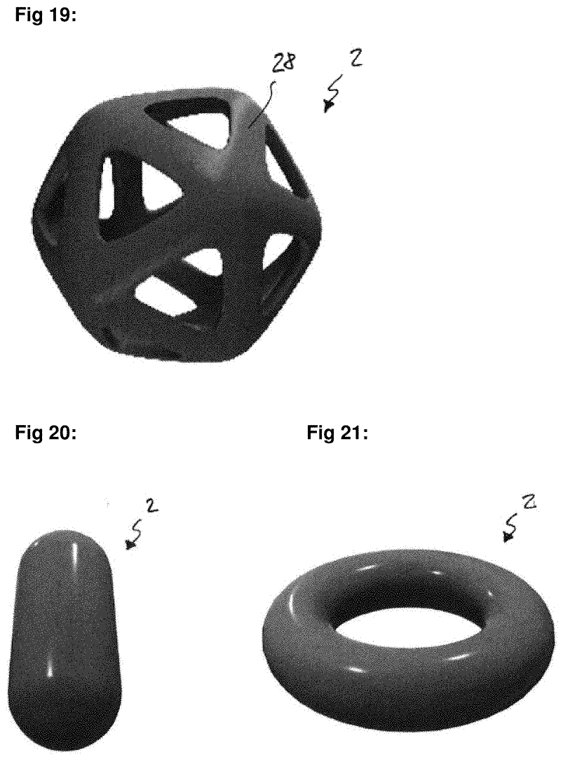

To prepare a solid administration form as depicted in FIGS. 5 and 6 an instrumental printer setup with two nozzles is used. Printing properties of both suitable thermal binders have to be evaluated before alternate printing using both nozzles.

Tablet dimensions planed with a total diameter of 10 mm and height of 4 mm containing a core of API mixture with a diameter of 5 mm and a height of 2 mm:

As properties of the first nozzle, the printing of pure PVA as suitable thermal binder prepared in example 1, same results used as evaluated for example 7:

As suitable thermal binary binder (PVA+20% Dipyridamole) printed by using the second nozzle material, prepared in Example 6, is pre-dried before feeding into the printing device. The residual moisture (goal<0.5%) is measured with an Aquatrac gauge at a temperature of 120° C. with 0.44%.

Using the preconditioned granulated material, prepared in Examples 1 and 6, neither bridging nor feeding problems are observed throughout the experimental series
Evaluation of printing parameter (second nozzle) and printing of solid administration form:
Determination of processing parameters and discharge properties

Granulated material prepared in Example 6 forms well separable droplets, homogeneously dropping out from the nozzle. At a nozzle temperature of 200° C. the material shows translucent droplets. The required drop height of 200 μm+10-20% is achieved with 60% discharge.
Conditions used for the printing process:

Temperature discharge unit: 190° C.

Temperature zone 2: 180° C.

Temperature zone 1: 170° C.

Temperature printing room: 80° C.

Dynamic pressure: 80 bar

Metering stroke: 5 mm

Decompression speed: 2 mm/s

Decompression space: 5 mm

Discharge: 60%

In order to find the suitable aspect ratio, test printings with different slicer volume (ratio of width and layer thickness) are adjusted. Best properties can be achieved with an aspect ratio of 1.32 using material prepared in Example 6.

By using conditions as described before, an optimized 3D printing process is performed with suitable binder of Example 1 (pure polyvinyl alcohol) for the outer part of the solid administration form. The core containing a mixture of PVA and 20% Dipyridamole (Example 6) is printed by the second nozzle. Using the set-up a solid administration form as projected and depicted in FIGS. 5 and 6 is printed.

FIG. 5 illustrates a schematic perspective view of one embodiment of a solid administration form. FIG. 6 illustrates a section view of the solid administration form shown in FIG. 5 along the line VI-VI in FIG. 5.

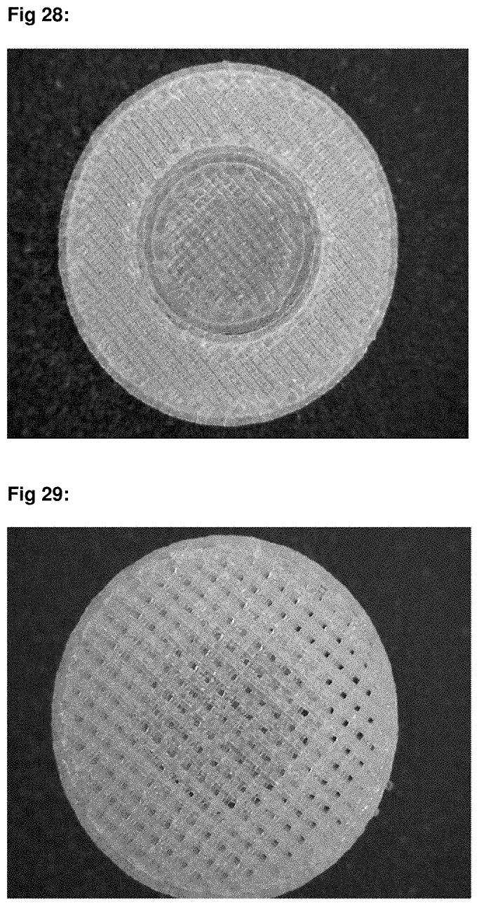

Resulting solid administration form with 100% filling rate containing in the outer part pure PVA and an inner core of a binary dispersion PVA as suitable thermal binder and 20% Dipyridamole (yellow color) as active pharmaceutical ingredient is analyzed by an optical method (FIG. 28).

### Example 14

### 3D Printing of Tablets with Outer Shell (50% Filling Rate) of Pure PVA and an Inner Core of a Binary Dispersion PVA as Suitable Thermal Binder and Dipyridamole (Yellow/Orange Color) as Active Pharmaceutical Ingredient

To prepare the solid administration form an instrumental printer setup with two nozzles is used. The printing properties of both suitable thermal binders have to be evaluated before alternate printing using both nozzles.

Tablets with tablet dimensions having a total diameter of 10 mm and height of 4 mm containing a core of an API mixture with a diameter of 5 mm and a height of 2 mm are prepared.

Same parameters are set for the first nozzle as found in the evaluation of example 7 for printing of pure PVA, as prepared in example 1 as suitable thermal binder.

As properties of the second nozzle, for printing of pure PVA+20% Dipyridamole as suitable binary thermal binder as prepared in example 6, same parameters are set as found in the evaluation of example 13.

By using conditions described before, an optimized 3D printing process is performed with 50% filling rate of the suitable binder of Example 1 (pure polyvinyl alcohol) for the outer part of the solid administration form. The core containing of a mixture of PVA and 20% Dipyridamole (Example 6) is printed with 100% filling rate by the second nozzle.

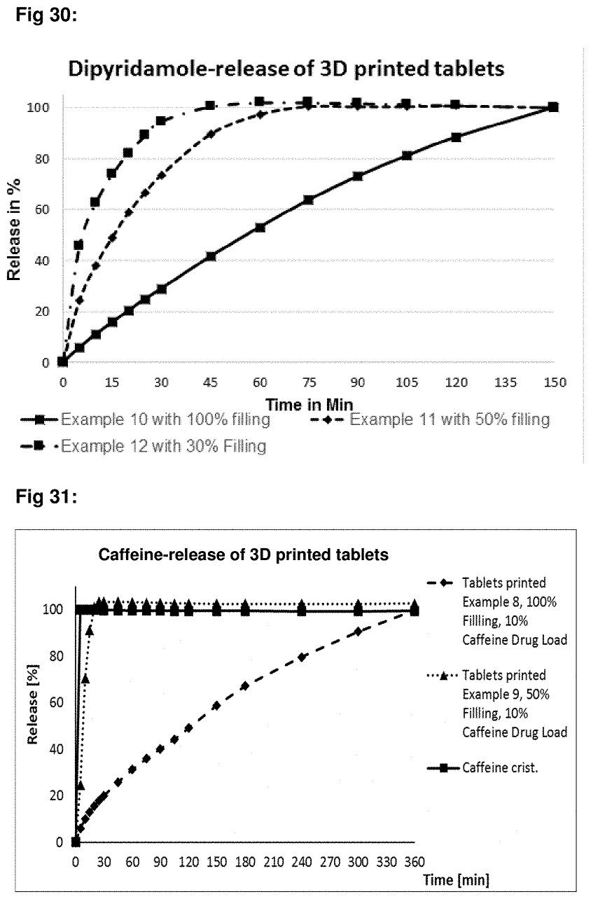

The resulting solid administration form with 50% filling rate containing in the outer part pure PVA and having an inner core with 100% filling rate of a binary dispersion of PVA as suitable thermal binder and 20% by weight of Dipyridamole (yellow color) as active pharmaceutical ingredient is analyzed by an optical method (FIG. 29).

### Analytical Evaluation (Dissolution) of Tablets Prepared by 3D Printing Process

Release of dipyridamole as active ingredient is determined using the Sotax Freisetzungsapparatur Sotax AT 7smart (Sotax AG, Lörrach, Germany)

The release determinations are carried out using Phosphate buffer pH 6.8 (900 ml) as the dissolution medium while stirring (paddle speed: 50 rpm) and measuring the absorbance with online UV-spectroscopy at 298 nm using 10 mm Cuvette.

Each sample is collected in a test tube with the automatic sampler.

### Release of Active Ingredient (Sotax)

Device: Release apparatus: Sotax AT 7smart (Sotax AG, Lörrach, Germany), Photometer Agilent 8453 (Agilent Technologies, Waldbronn, Germany)

Number of vessels: 6

Method: Paddle

Medium: Phosphate buffer pH 6.8

Amount of medium: 900 mL

Temperature of medium: 37° C.

Rotation: 50 rpm

Duration: 2 h

Time of sampling: 5, 10, 15, 20, 25, 30, 45, 60, 75, 90, 105, 120 min

Final spin: no

Cuvette layer thickness: 10 mm

Wavelength: 289 nm

FIG. 30 illustrates results achieved by dissolution measurement of 3D printed dipyridamole containing tablets in 900 ml of phosphate buffer pH 6.8. The release study comparing different filling rate of the 3D printed tablets (Example 10=100% Tablet Filling rate/Example 11=50% Tablet Filling rate/Example 12=30% Tablet Filling rate) shows substantial differences in the release of the active ingredient (dipyridamole). To dissolute and release the full API amount of an 100% filled tablet 150 minutes measured, while a 50% filled 3D printed tablet already releases 100% of its API amount after approximately 60 minutes in the dissolution equipment. As expected, a 30% filled 3D printed tablet dissolved much faster and 100% release of its API amount could be achieved after app 30 minutes of test time.

### Standardized Release of 3D-Printed Tablets (Dipyridamole) in PP, pH 6.8

### Release 3D-Printed Tablets (Caffeine) in 0.1 M HCl

### FIG. 31

### Analytical Evaluation (Dissolution) of Tablets Prepared by 3D Printing Process.

Release of caffeine as active ingredient is determined using the Sotax Freisetzungsapparatur Sotax AT 7smart (Sotax AG, Lörrach, Germany)

Phosphate buffer pH 6.8 (900 ml) was used as the dissolution medium with 50 rpm, paddle speed and the release determinations are carried out with online UV, 298 nm 10 mm Cuvette

Each sample is collected in a test tube with the automatic sampler.

### Release of Active Ingredient (Sotax)

Device: Release apparatus: Sotax AT 7smart (Sotax AG, Lörrach, Germany), Photometer Agilent 8453 (Agilent Technologies, Waldbronn, Germany)

Number of vessels: 6

Method: Paddle

Medium: 0.1 M HCl

Amount of medium: 900 mL

Temperature of medium: 37° C.

Rotation: 100 rpm

Duration: 6 h

Time of sampling: 5, 10, 15, 20, 25, 30, 45, 60, 75, 90, 105, 120, 150, 180, 240, 300, 360 min

Final spin: no

Cuvette layer thickness: 10 mm

Wavelength: 272 nm

FIG. 31 illustrates results achieved by dissolution measurement of 3D printed caffeine containing tablets in 900 ml of 0.1 n HCl. The release study compares different filling rates of the 3D printed tablets (Example 8=100% Tablet Filling rate/Example 9=50% Tablet Filling rate) and shows substantial differences in the release of the active ingredient (caffeine). To dissolute and release the full API amount of a filled tablet (100%) needs 360 minutes for entire release of the comprising API, while a 3D printed tablet, 50% filled, already releases 100% of the comprising API amount after app 30 minutes in the dissolution equipment. The time measured is not much faster than dissolving pure crystalline caffeine particles tested in comparison by 100% after app 5 minutes.

1. A method for manufacturing a solid administration form (2) comprising at least one active pharmaceutical ingredient, wherein a flowable but setting composite material (16, 20, 25, 26, 27) comprising the at least one active pharmaceutical ingredient is added together and set to generate the solid administration form (2), characterized in that the flowable composite material (16, 20, 25, 26, 27) is liquefied and delivered to at least one discharge unit (3), and that small portions (12) of the liquefied composite material (16, 20, 25, 26, 27) are intermittently discharged through an outlet of the discharge unit (3) into a setting unit (13) where the setting of the small portions (12) occurs, thereby gradually generating the solid administration form (2).

2. The method of claim 1, characterized in that the flowable composite material (16, 20, 25, 26, 27) comprises a polymer or combination of different polymers and at least one amorphous active pharmaceutical ingredient that is dispersed or dissolved within the polymer.

3. The method of claim 1, characterized in that the flowable but setting composite material (16, 20, 25, 26, 27) includes non-soluble porous or non-porous carrier particles for altering or enhancing the properties of the solid administration form (2).

4. The method of claim 1, characterized in that the flowable composite material (16, 20, 25, 26, 27) is fabricated during delivery to the discharge unit (3).

5. The method of claim 1, characterized in that the flowable composite material (16, 20, 25, 26, 27) is made of or comprises granules prepared by known methods like e.g. hot melt extrusion, wet granulating, dry compaction or twin screw granulation or/and a particle kind of material.

6. The method of claim 1, characterized in that the small portions (12) of the liquefied composite material (16, 20, 25, 26, 27) are droplets and that the solid administration form (2) is generated by adding droplets that bond or stick together before or during the setting of the liquefied composite material (16, 20, 25, 26, 27).

7. The method of claim 6, characterized in that an average diameter of the droplets is less than 350 μm, and in that the average diameter of the droplets is larger than 20 μm.

8. The method of claim 1, characterized in that there is a void space (14, 24) between at least some small portions (12) that are placed adjacent to each other, resulting in a porous structure of the solid administration form (2).

9. The method of claim 1, characterized in that before or after discharging a predetermined first amount of a composite material (16) a predetermined second amount of a second material (18) is discharged, whereby the material of the second material (18) differs from the composite material (16).

10. The method of claim 1, characterized in that composite material (16, 20, 25, 26, 27) is discharged from more than one discharge units (3), which have different sizes.

11. The method of claim 1, characterized in that the small portions (12) of the composite material (16, 20, 25, 26, 27) are discharged into an arrangement of the small portions (12) such that the solid administration form (2) comprises at least two regions with different characteristics of the active pharmaceutical ingredient and optionally different porosity.

12. Solid administration form (2) comprising at least one active pharmaceutical ingredient, whereby the solid administration form (2) is manufactured by liquefying at least one flowable composite material (16, 20, 25, 26, 27) and delivering the liquefied composite material(s) (16, 20, 25, 26, 27) to at least one discharge unit (3), whereby small portions (12) of the liquefied composite material are intermittently discharged through the outlet(s) of the discharge unit(s) (3) into a setting unit where the setting of small portions (12) occurs, thereby gradually generating the solid administration form (2) by performing the method of claim 1.

13. The method of claim 7, wherein the average diameter of the droplets is less than 200 μm.

14. The method of claim 7, wherein the average diameter of the droplets is larger than 50 μm.

15. The method of claim 13, wherein the average diameter of the droplets is larger than 50 μm.
